(19) United States
(12) Patent Application Publication
(10) Pub. No.: US 2023/0255212 A1
(43) Pub. Date: Aug. 17, 2023

(54) Use of Paperclip RNA Structure to Inhibit Target Gene Expression

(71) Applicant: University of Manitoba, Winnipeg (CA)

(72) Inventor: Steve Whyard, Winnipeg (CA)

(21) Appl. No.: 18/005,118

(22) PCT Filed: Jul. 5, 2021
(86) PCT No.: PCT/CA2021/050911

§ 371 Date: Jan. 11, 2023

Related U.S. Application Data
(60) Provisional application No. 63/052,056, filed on Jul. 15, 2020.

(51) Int. Cl. A01N 63/60; C12N 15/113

(52) U.S. Cl. CPC A01N 63/60; C12N 15/113; C12N 2310/14; C12N 2310/531

(57) ABSTRACT

Described herein is a “paperclip” structured dsRNA (pcRNA) that has two closed ends (FIG. 1), and was developed and tested for its ability to enter insect cells and induce RNAi. While conventional dsRNAs, with their two collinear RNA strands, enter insect cells by clathrin-mediated endocytosis, the pcRNAs can enter cells by a clathrin-independent mechanism. The new structured dsRNA can be used to deliver dsRNA to insects that either develop resistance through alterations to their conventional uptake mechanisms and to insects that are naturally refractory to dsRNAs, including lepidopteran pests.

### PRIOR APPLICATION INFORMATION

The instant application claims the benefit of US Provisional Application 63/052,056, filed Jul. 15, 2020 and entitled “USE OF PAPERCLIP RNA STRUCTURE TO INHIBIT TARGET GENE EXPRESSION”, the entire contents of which are incorporated herein by reference for all purposes.

### BACKGROUND OF THE INVENTION

Our heavy reliance on chemical pesticides has resulted in increased incidences of insecticide resistance in pest insect populations and increasing negative impacts on non-target species. A new generation of species-limited insecticides based on double-stranded RNA is being developed by an increasing number of research groups worldwide [1]. Delivery of double-stranded RNA to an organism will result in sequence-specific mRNA degradation in a process known as RNA interference (RNAi). By delivering a double-stranded RNAs that target gene(s) critical for growth or development of the insect, it is possible to kill the insect by selectively reducing the targeted gene's expression [2, 3].

While dsRNAs may be viewed as a new generation of environmentally friendly pesticides, some of our most serious agricultural pests, including the majority of lepidopteran pest insects (moths), are refractory to dsRNA. When these insects ingest dsRNAs, their gut cells fail to deliver the ingested dsRNA to the target mRNA molecules. Hence, dsRNA-based insecticides will remain largely ineffective against these insects unless improvements to dsRNA delivery can be achieved. DsRNA pesticides are poised to reach our markets shortly, and could protect many of our crops without adversely affecting non-target, beneficial species. While it will be relatively easy to design a large repertoire of dsRNAs to target essential genes in insects, one type of resistance that could seriously affect the versatility of dsRNA-based pesticides is reduced dsRNA uptake. Recently, a group of researchers developed a lab-bred dsRNA-resistant strain of corn rootworm that displayed a greatly reduced ability to absorb dsRNAs into their gut cells, as discussed below.

Different insect species display differential responses to exogenously applied dsRNAs. Some insects, including many beetles, are highly sensitive to dsRNAs, requiring only small quantities of dsRNAs to kill the insect. Other insects, in particular lepidopteran species (butterflies and moths), can be highly refractory to double-stranded RNA, and fail to show any RNAi-mediated transcript knockdown unless very high doses of dsRNAs are delivered [4]. One factor that can strongly impact RNAi efficacy in an insect is the ability of the dsRNAs to enter targeted cells and to disperse throughout the cytoplasm to reach the targeted mRNA molecules. In some RNAi-refractory lepidopterans, the inability of the dsRNAs to reach the cytoplasm of the gut cells of the feeding insects is considered a primary reason why these insects fail to respond to ingested dsRNAs [5].

Even in highly RNAi-sensitive insects such as the coleopteran corn rootworm, resistance to dsRNA cellular uptake has been observed in a lab strain that was subjected to increasing concentrations of dsRNA over 11 generations [6], as discussed above. In this lab selected strain, the insects ingested more than 150 times the normal dose of double-stranded RNA required to kill susceptible corn rootworms, without showing any adverse effects. Although the precise mechanism of the resistance has not been fully elucidated, it was observed that the dsRNAs failed to enter the gut cells of these resistant insects. This finding suggests that dsRNA uptake mechanisms, if placed under strong selection pressures, could reduce the efficacy of dsRNA-based insecticides.

DsRNA uptake has been examined in a broad range of invertebrates, and two primary mechanisms have been observed: 1) RNA transport proteins; and 2) clathrin-mediated endocytosis [7]. In the nematode Caenorhabditis elegans, ingestion of dsRNAs results in highly effective RNAi, and the uptake and spread of the dsRNAs is mediated by several systemic RNAi-deficient (SID) RNA transport proteins [8]. Putative homologues of the C. elegans SID-1 protein have been identified in several insect species but their role in dsRNAs uptake can vary. In the corn rootworm (D. virgifera), for example, SID-like proteins (SIL) appear to contribute to dsRNAs uptake [9], but in the red flour beetle (Tribolium castaneum), knockdown of these SIL proteins had no negative effect on double-stranded RNA uptake [10], suggesting that the SIL proteins do not have a significant role in mediating RNAi in this insect, and that other dsRNA uptake mechanisms are contributing to dsRNA in this insect.

In many invertebrates, including the nematode C. elegans and the corn rootworm, a major contributor of dsRNA uptake is clathrin-mediated endocytosis (CME) [11]. In this process, dsRNA would bind to an as yet unidentified cell surface receptor. This binding would result in the recruitment of intracellular clathrin proteins, and together with other accessory proteins, will form an endocytic vesicle. The endocytic vesicles will merge together to form early endosomes, which, following recruitment of other proteins will mature into late endosomes. In the few insects examined to date, the dsRNA appears to escape the endosomes to enter the cytoplasm before complete maturation of the late endosomes [7].

In most insect RNAi applications, dsRNA is administered to cells or insects as long (>200 bp) dsRNA. Short dsRNAs (<60 bp) are generally regarded as ineffective inducers of RNAi, as they have failed to be taken up by gut cells in various insects [12]. However, short hairpin RNAs (shRNAs) have been used successfully in a limited number of insects [13,14]. With these shRNAs, a single-stranded dsRNA is folded over on itself to create a short (typically 21-25 bp) double-stranded RNA structure with a single-stranded closed-loop. These shRNAs are generally considered more stable, and potentially more resistant to denaturation then the conventional linear dsRNA molecules.

### SUMMARY OF THE INVENTION

According to a first aspect of the invention, there is provided a synthetic RNA molecule that, following synthesis, folds to form a secondary structure comprising:

a) a double stranded region comprising an RNAi sequence of at least 21 nucleotides;

b) two single-stranded hairpin loops flanking either end of the double-stranded region; and

c) a 5′ end and a 3′ end within the double stranded region that are closed but not covalently sealed.

According to another aspect of the invention, there is provided a method of reducing feeding damage to a plant from a biological pest of interest comprising:

applying to the plant to be protected from the biological pest of interest an effective amount of a synthetic RNA molecule to at least a portion of the plant to be protected from the biological pest of interest, the synthetic RNA molecule folding to form a secondary structure following synthesis that comprises:

a) a double stranded region comprising an RNAi sequence specific for the biological pest of interest, said RNAi sequence being at least 21 nucleotides;

b) two single-stranded hairpin loops flanking either end of the double-stranded region; and

c) a 5′ end and a 3′ end within the double stranded region that are closed but not covalently sealed,

said synthetic RNA molecule reducing feeding damage to the plant to be protected by reducing feeding activity of the biological pest of interest following ingestion of said synthetic RNA molecule by the insect of interest.

According to a further aspect of the invention, there is provided a method of protecting a plant from feeding damage from a biological pest of interest comprising:

applying to the plant to be protected from the biological pest of interest an effective amount of a synthetic RNA molecule to at least a portion of the plant to be protected from the biological pest of interest, the synthetic RNA molecule folding to form a secondary structure following synthesis that comprises:
a) a double stranded region comprising an RNAi sequence specific for the biological pest of interest, said RNAi sequence being at least 21 nucleotides;
b) two single-stranded hairpin loops flanking either end of the double-stranded region; and
c) a 5′ end and a 3′ end within the double stranded region that are closed but not covalently sealed,

said synthetic RNA molecule reducing feeding damage to the plant to be protected by reducing feeding activity of the biological pest of interest following ingestion of said synthetic RNA molecule by the insect of interest.

According to yet another aspect of the invention, there is provided a method of killing a biological pest of interest comprising:

applying to at least a portion of a food source of the biological pest of interest an effective amount of a synthetic RNA molecule, the synthetic RNA molecule folding to form a secondary structure following synthesis that comprises:
a) a double stranded region comprising an RNAi sequence specific for the biological pest of interest, said RNAi sequence being at least 21 nucleotides;
b) two single-stranded hairpin loops flanking either end of the double-stranded region; and
c) a 5′ end and a 3′ end within the double stranded region that are closed but not covalently sealed,

said synthetic RNA molecule killing the biological pest of interest following ingestion of said synthetic RNA molecule by the biological pest of interest.

### BRIEF DESCRIPTION OF THE DRAWINGS

FIG. 1. Schematic drawing of an exemplary short paperclip RNAs (pcRNAs), with two closed ends, but not covalently sealed. The “missing” phosphodiester bond is highlighted with an asterisk. Image produced using RNAfold program, with base-pairing probabilities scored using the heat map. Nucleotides labelled red indicate high certainty of predicted secondary structure.

### DESCRIPTION OF THE PREFERRED EMBODIMENTS

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are now described. All publications mentioned hereunder are incorporated herein by reference.

As discussed herein, we have developed dsRNAs, both long and short, that are sealed on both ends to create what we are calling a “paperclip” dsRNA, otherwise known as a pcRNA. A previously described covalently closed dsRNA molecule proved ineffective at producing RNAi, as it failed to be processed by the enzyme Dicer [15], which is responsible for dicing exogenous dsRNAs into the effector 21 nt-long siRNAs that mediate RNAi. In contrast, the pcRNAs are not completely closed circles, but rather, they possess two free ends (3′ and 5′), a feature that facilitates efficient processing by Dicer and enables effective RNAi. Aside from this structural difference, unlike long linear dsRNAs, siRNAs, and hpRNAs, which enter insect cells by CME, the pcRNAs are capable of entering the cells by a clathrin-independent mechanism.

Although the molecular mechanism of this clathrin-independent uptake process has not been identified, the fact that the pcRNAs can enter insect cells by an alternative mechanism allows for the delivery of dsRNAs to insects that have either developed uptake resistance or they are naturally recalcitrant to uptake of double-stranded RNA. Furthermore, while not wishing to be bound to a particular theory or hypothesis, it is believed that the pcRNA may be entering the cell by caveolin-dependent endocytosis; lipid-raft-dependent endocytosis; or by an unidentified dsRNA transporter, perhaps distantly related to SID proteins.

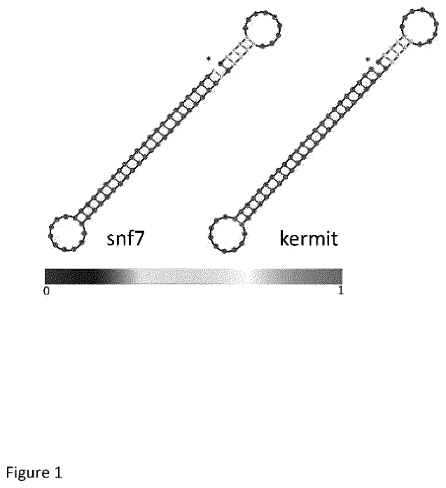

As discussed herein, this “paperclip” structured dsRNA (pcRNA), shown schematically in FIG. 1 has two closed ends (FIG. 1), and was developed and tested for its ability to enter insect cells and induce RNAi. While conventional dsRNAs, with their two collinear RNA strands, enter insect cells by clathrin-mediated endocytosis, the pcRNAs can enter cells by a clathrin-independent mechanism, as discussed above. This alternative structured dsRNA can readily enter lepidopteran cells. The new structured dsRNA can therefore be used to deliver dsRNA to insects that either develop resistance through alterations to their conventional uptake mechanisms and to insects that are naturally refractory to dsRNAs, including lepidopteran pests.

This dsRNA has been developed as an alternative to conventional (linear) dsRNAs for insecticidal applications. Like other dsRNAs, it can be designed to be species-specific, thereby providing pest insect control without adversely affecting non-target species. Specifically, as discussed herein, the pcRNA includes an RNAi sequence of at least 21 nucleotides that is capable of interfering with expression of a target gene. As will be known by those of skill in the art, the target gene selected is typically a gene whose expression is critical for growth and/or development of the target organism.

Examples of suitable gene targets will be readily apparent to one of skill in the art and are within the scope of the invention. However, by way of example, suitable targets may include but are by no means limited to: a) genes involved in insect cuticle formation (for example, chitin synthase; chitinase); b) genes involved in control of metamorphosis (for example, ecdysone receptor; ultraspiracle; prothoracicotropic hormone; molting defective); c) genes involved in cellular respiration (for example cytochrome c heme lyase, superoxide dismutase; catalase); d) genes involved in DNA structure/organization (for example, dre4, ssrp, condensin); e) genes involved intracellular transport (for example, Snf7; Rab6; dynamin); and f) genes involved in neural function (for example, Fez2; Ace). Alternatively, the target gene may be a gene targets for producing sterile male insects. As will be apparent to one of skill in the art, these gene targets (for example, Tssk1, Vasa, Doublesex, Zpg, and gas8) may be used for sterile insect technique applications.

As discussed herein, pcRNAs enter insect cells by a clathrin-independent manner, unlike other conventional dsRNAs that use clathrin-mediated endocytosis as the main mode of cellular entry. pcRNAs can therefore provide an alternative dsRNA to overcome dsRNA uptake resistance, which has already been demonstrated in lab-selected insects.

Furthermore, as discussed herein, pcRNAs enter lepidopteran (moth) cells and induce RNAi, unlike conventional dsRNAs, which generally fail to induce RNAi in this large group of ecumenically-important pests. Hence, these new molecules can be used to control pests that are currently refractory to dsRNA.

Described herein is a synthetic RNA molecule that, following synthesis, folds to form a secondary structure comprising:

a) a double stranded region comprising an RNAi sequence of at least 21 nucleotides;

b) two single-stranded hairpin loops flanking either end of the double-stranded region; and

c) a 5′ end and a 3′ end within the double stranded region that are closed but not covalently sealed.

As will be appreciated by one of skill in the art, the synthetic RNA molecule is a single stranded RNA in which both ends fold over on itself to create a perfectly duplexed double-stranded RNA, but lacking a phosphodiester bond where the 5′ and 3′ ends meet.

As discussed herein, the RNAi sequence is a nucleotide sequence that is specific for a target gene of biological subject of interest.

In some embodiments, the biological subject of interest is a biological pest, for example, an insect.

As discussed herein, the synthetic RNA molecule of the invention is designed to fold such that the 5′ end and the 3′ end are proximal to one another and are separated by at least one phosphodiester bond. As will be apparent to one of skill in the art, if the “missing” phosphodiester bond that corresponds to the gap between the 5′ end and the 3′ end of the synthetic RNA molecule was present, the synthetic RNA molecule would be covalently closed.

As discussed herein, the folded RNA molecule is arranged to fold spontaneously such that the 5′ end and the 3′ end are separated by a gap corresponding to the length of at least one phosphodiester bond.

The fact that the 5′ end and the 3′ end are proximal but not covalently closed, that is, are separated by at least a missing phosphodiester bond is important as closed circular dsRNA are not effectively processed into siRNAs (the effector molecules of RNAi) by Dicer [15].

Furthermore, as discussed herein, the synthetic RNA molecule of the invention is designed such that the gap corresponding to at least the missing phosphodiester bond is positioned such that the double stranded region comprises at least 23 nt on the 5′ side of the gap. As will be apparent to one of skill in the art, these at least 23 nt of the double stranded RNA region correspond to the RNAi sequence. Furthermore, this arrangement allows for Dicer to generate a 21 bp siRNA, as discussed herein. The two extra nucleotides ensure that Dicer can cut dsRNA efficiently.

The other side or 3′ side of the gap or the “missing” phosphodiester bond is, as discussed herein, a minimum of 3 nt to ensure stability of the 3′ end. That is, at least 3 nt are required so that the paperclip structure is held securely as well as so that the secondary structure of the synthetic RNA molecule forms as desired when folding spontaneously.

As will be apparent to one of skill in the art, as used herein, “spontaneously” in regards the folding of the synthetic RNA molecule refers to the fact that it is not necessary to add any additional factors or agents known in the art to promote proper folding of the synthetic RNA molecule so that the desired RNA secondary structure forms.

As will be appreciated by one of skill in the art, the synthetic RNA molecule may be of any suitable length. As will be known by those of skill in the art, typically, dsRNAs up to 300 bp are effective at knocking down transcripts, anything larger is not usually found to be any more effective, and there is greater potential for synthesis errors, thereby reducing the overall effectiveness of the synthetic RNA molecule.

Furthermore, as discussed herein, while not wishing to be bound to a particular theory or hypothesis, it is believed that relatively smaller pcRNAs may be binding more efficiently to the endocytic protein machinery or that larger pcRNAs may not hold together as firmly, as the dsRNA regions may open and close in places, leaving them subject to refolding or possible endonuclease attack.

As discussed above, the length of the double stranded RNA region is at least 23 nucleotides on a 5′ side of the gap. That is, in the double stranded region of the synthetic RNA molecule when the synthetic RNA molecule is folded, there is at least a 23 nt double stranded region starting at the 5′ end of the synthetic RNA molecule.

Dicer will generate 21 nt short interfering RNAs (siRNAs) from any length of dsRNA >21 nt in length. Specifically, Dicer slides along a double-stranded RNA molecule and cuts at 21 nt intervals. Hence, a double stranded region comprising 42 nt would be cut into two siRNA molecules by Dicer, while a 100 bp dsRNA would produce 4 siRNAs. Once the siRNA molecules have been generated, the RNA interference machinery [e.g. RISC] in the cell is able to use the siRNA to impair expression of the target gene as discussed herein. Accordingly, while the RNAi sequence is at least 23 nts, it may be longer, for example, 44 nts, 65 nt, 86 nts, 107 nt, 128 nt, 149 nts, for generation of multiple, different siRNA molecules, as discussed herein. Furthermore, this 5′ end double stranded region which comprises the RNAi sequence may be any suitable length, bearing in mind synthesis fidelity and mis-folding concerns as will be apparent to those of skill in the art and as discussed herein.

As discussed above, the length of the double stranded region of the synthetic RNA molecule of the invention starting with the 3′ end of the synthetic RNA molecule is at least 3 nucleotides, that is, there is at least 3 nt of double stranded RNA on a 3′ side of the gap. As discussed above, this 3 nt sequence is the minimum sequence required from stability of the RNA molecule of the invention, specifically, so that the desired secondary structure forms spontaneously and stably. As will be appreciated by one of skill in the art, this 3′ end double stranded region may be of any suitable length, as greater lengths will increase stability of the synthetic RNA molecule when folded, bearing in mind synthesis fidelity and mis-folding concerns as will be apparent to those of skill in the art and as discussed herein.

As will be apparent to one of skill in the art on consultation with at least FIG. 1, the RNA sequences that complementarily bind together to form the double stranded region are each separated by a hairpin loop, wherein each hairpin loop independently comprises 3-15 nucleotides, 3-12 nucleotides, 6-15 nucleotides, or 6-12 nucleotides.

As will be appreciated by one of skill in the art, the hairpin loops can be variable in size. For example, loops as short a 3 nt may be used in some hairpins, but these short loops may not always fold over 100% of the time. Loops between 6 and 15 nt are often used for hairpins we tested loops of 6 nts, 9 nts, and 12 nts and observed that 9 bp worked consistently. Specifically, while all worked equally well, 9 nt loops gave more consistent knockdown. Ideally, the loop should be kept as small as possible to make it easy to synthesize. Furthermore, while not wishing to be bound to a particular theory or hypothesis, it is believed that loops longer than 15 nts, for example, longer than 12 nt, can potentially generate their own internal secondary structure, and were avoided.

According to another aspect of the invention, there is provided a method of reducing feeding damage to a plant from a biological pest of interest comprising:

applying to the plant to be protected from the biological pest of interest an effective amount of a synthetic RNA molecule to at least a portion of the plant to be protected from the biological pest of interest, the synthetic RNA molecule folding to form a secondary structure following synthesis that comprises:

a) a double stranded region comprising an RNAi sequence specific for the biological pest of interest, said RNAi sequence being at least 21 nucleotides;

b) two single-stranded hairpin loops flanking either end of the double-stranded region; and

c) a 5′ end and a 3′ end within the double stranded region that are closed but not covalently sealed,

said synthetic RNA molecule reducing feeding damage to the plant to be protected by reducing feeding activity of the biological pest of interest following ingestion of said synthetic RNA molecule by the insect of interest.

The synthetic RNA molecule may be co-administered with a nuclease inhibitor.

Alternatively, the synthetic RNA molecule of the invention may be co-administered with a second synthetic RNA molecule, for example, a synthetic RNA molecule of the invention comprising a different RNAi sequence than the first synthetic RNA molecule. Alternatively, the second synthetic RNA molecule may be a dsRNA molecule such as those known in the prior art for generating siRNA transcripts.

According to another aspect of the invention, there is provided a method of protecting a plant from feeding damage from a biological pest of interest comprising:

applying to the plant to be protected from the biological pest of interest an effective amount of a synthetic RNA molecule to at least a portion of the plant to be protected from the biological pest of interest, the synthetic RNA molecule folding to form a secondary structure following synthesis that comprises:
a) a double stranded region comprising an RNAi sequence specific for the biological pest of interest, said RNAi sequence being at least 21 nucleotides;
b) two single-stranded hairpin loops flanking either end of the double-stranded region; and
c) a 5′ end and a 3′ end within the double stranded region that are closed but not covalently sealed,

said synthetic RNA molecule reducing feeding damage to the plant to be protected by reducing feeding activity of the biological pest of interest following ingestion of said synthetic RNA molecule by the insect of interest.

According to another aspect of the invention, there is provided a method of killing a biological pest of interest comprising:

applying to at least a portion of a food source of the biological pest of interest an effective amount of a synthetic RNA molecule, the synthetic RNA molecule folding to form a secondary structure following synthesis that comprises:
a) a double stranded region comprising an RNAi sequence specific for the biological pest of interest, said RNAi sequence being at least 21 nucleotides;
b) two single-stranded hairpin loops flanking either end of the double-stranded region; and
c) a 5′ end and a 3′ end within the double stranded region that are closed but not covalently sealed,

said synthetic RNA molecule killing the biological pest of interest following ingestion of said synthetic RNA molecule by the biological pest of interest.

As will be appreciated by one of skill in the art, an “effective amount” as used herein refers to an amount that is sufficient to achieve the desired result, that is, to reduce feeding activity of the biological pest of interest or to reduce damage to a plant from a biological pest of interest compared to an untreated control plant of similar size, age and type or to a food source of the biological pest of interest to kill the biological pest of interest. As such, an “effective amount” may depend on several factors, such as the environmental conditions, weather conditions, and the number of the biological pest of interest that the plant or food source may encounter or is expected to encounter, which can be estimated using any of a variety of means known in the art.

As discussed herein, the synthetic RNA molecule of the invention may be applied to at least one leaf of the plant to be protected or to at least a portion of the food source. The synthetic RNA molecule may be applied to the plant to be protected or to the food source at a concentration of at least about 0.1 ng per mm², or at least about 0.5 ng per mm², that is, per mm² of plant material being coated, for example, one or more leaves.

As discussed herein, the synthetic RNA molecule of the invention was applied onto the leaves in just water. However, it is of note that there are a variety of formulation additives known in the art that act for example as spreaders, stickers and penetrants when applied to plants or other food sources, for example, to leaves of plants. As one of skill in the art will appreciate, such formulation additives can be tested and optimized by one of routine skill in the art and are within the scope of the invention.

In some embodiments of the invention, the effective amount of the synthetic RNA molecule of the invention is co-administered with an effective amount of a nuclease inhibitor.

Examples of known nuclease inhibitors include but are by no means limited to strong protein denaturants, such as guanidinium isothiocyanate; anionic polymers such as polyvinylsulfonic acid and protein-based nuclease inhibitors, such as: GamS Nuclease Inhibitor; Superase protein-based RNase inhibitor; and Recombinant RNase Inhibitor.

The invention will now be further explained and/or elucidated by way of examples; however, the invention is not necessarily limited to or by the examples.

### Example 1—PcRNAs can Induce RNAi when Delivered to Insect Cells

Two genes were targeted for RNAi-mediated knockdown in cultured Aedes aegypti mosquito cells: snf7 and kermit. To produce long linear dsRNAs, gene fragments of approximately 200 bp were PCR-amplified from mosquito cDNA. All PCR primers contained T7 sequences to facilitate RNA synthesis in in vitro transcription reactions, to produce dsRNA. A list of the dsRNAs used is provided in Table 1. For the other dsRNAs (siRNAs, long/short hpRNAs, and long/short pcRNA), DNA oligonucleotides, also with T7 linkers, were purchased from IDT, ligated by slow cooling (from 95oC down to 25° C.), and then used directly in in vitro transcription reactions.

Mosquito cells were transfected with the different lengths and structures of dsRNA using Lipofectamine-3000 transfection reagent, to evaluate their ability to knock down the targeted genes if delivered directly into the cells. The long dsRNAs targeting Snf7 and kermit were 204 and 213 bp, respectively, whereas the siRNA, short hpRNA, and short pcRNA each contained a contiguous 23 bp long dsRNA section. For the short dsRNAs, a 10× higher concentration of each was delivered to the cells, to provide approximately the same number of siRNA to the cells as the long dsRNA would generate from dicer processing. The values shown in Table 2 represent the means and standard errors of 10 replicate well treatments. No significant difference in % knockdown was observed for any of the different dsRNA treatments (ANOVA, p >0.7 for all comparisons).

As can be seen in Table 2, all of the dsRNAs, regardless of the length or shape, could mediate target transcript knockdown, as assessed qRT-PCR analyses. For the qRT-PCR analyses, two reference genes, actin and RpS7, were used to normalize the transcript levels in the different treatments. Using the transfection reagent to deliver the dsRNAs to the mosquito cells resulted in similar levels of knockdown of each of the two transcripts, regardless of their lengths or structure. This result indicates that the pcRNA is a suitable substrate for Dicer and the other RNAi machinery, for both the long and short pcRNA structures.

Two genes were similarly targeted for RNAi mediated knockdown in cultured Sf9 fall armyworm cells: actin and the putative orthologue of kermit. Using the same approach, knockdown of each gene was achieved using each of the different lengths and structures of dsRNA in this lepidopteran cell line, indicating that the RNAi machinery is functional and that the pcRNA could also be used as an effective substrate for dicer and the other RNAi machinery, provided the dsRNAs were delivered by a transfection reagent. The results are shown in Table 3, which demonstrates that all dsRNA molecules were equally effective (when doses were adjusted to accommodate different numbers of siRNA generated by the long dsRNAs). All values represent the means and standard errors from 10 replicate wells of cells. The extent of knockdown was similar for all treatments (ANOVA, p>0.5)

### Example 2—Treatment of Mosquito Cells with Clathrin Inhibitors Prevents RNAi, Except when pcRNAs are Delivered

To examine DsRNA uptake in cultured Aedes aegypti cells, CCL125 cells were seeded to six well plates. Once cells and reached 80% confluence, culture media was withdrawn and cells were washed with PBS, and were then treated for 30 minutes to one of two clathrin inhibitors (0.2 μM bafilomycin-A1, 10 μM chlorpromazine), a micropinocytosis/phagocytosis inhibitor (10 μM cytochalsin-D), and a calveolae-dependent endocytosis inhibitor (2 mM methyl βcyclodextrin). The cells were washed with PBS and different lengths and structures of dsRNA targeting actin transcripts were added to the media. After two hours, the cells were washed to remove excess dsRNA, and 24 hours later the cells were harvested, RNA extracted, and the level of actin transcripts was evaluated by qRT-PCR. Table 4 shows the percent transcript knockdown of snf7 or kermit following exposure of the A. aegypti CCL-125 cells to different structured dsRNAs in the presence or absence of chlorpromazine. Values represent the means and standard errors of 5 replicate experiments consisting of 6 well each. Treatments demonstrating significantly impaired RNAi, relative to the “no inhibit control” are highlighted with an asterisk.

As can be seen in Table 4, in the absence of chlorpromazine, the long linear dsRNA, the long hpRNA, the long pcRNA, and the short pcRNA were equally effective at knocking down the targeted transcripts, whereas the siRNA and shRNA were less effective, but nevertheless still caused a knockdown. Only the clathrin inhibitors were observed to inhibit RNAi-mediated knockdown of actin, using long linear dsRNA, siRNA, and hpRNA. The other chemical inhibitors had no impact on the ability of the cells to take up double-stranded RNA and mediate RNAi. Interestingly, both the long and short pcRNAs were able to enter mosquito cells and induce RNAi even in the presence of the clathrin inhibitors. This finding indicates that CME is important for the uptake of long linear dsRNA, siRNAs, and hpRNAs but is not needed for uptake of pcRNA.

### Example 3—PcRNA can Enter Spodoptera (Sf9) Cells Whereas Linear or Hairpin dsRNAs Cannot

DsRNA targeting actin transcripts were applied to Spodoptera frugiperda (fall armyworm) Sf9 cells, and after two hours, the cells were washed and 24 hours later, the cells were harvested, RNA extracted, and qRT-PCR was used to assess knockdown of actin transcripts. Table 5 shows the percent knockdown of actin transcripts in Sf9 cells treated with different dsRNAs. The values represent the means and averages of 5 replicates of 4 wells of cells.

As can be seen in Table 5, the linear or hairpin dsRNAs failed to induce knockdown in the lepidopteran cells, which is not unexpected, given that these cells are known to be refractory to exogenous dsRNA. In contrast, the long and the short pcRNAs were able to enter the cells, but interestingly, the short pcRNAs were able to mediate a three-fold stronger knockdown of the actin transcripts. These findings indicate that the pcRNA structure facilitates entry into these lepidopteran cells, and the short pcRNAs appear to either enter the cells more readily or are processed more efficiently than the longer pcRNAs.

### Example 4—Treatment of Mosquito Larvae with the Clathrin Inhibitor Prevents RNAi, Unless pcRNAs are Applied

Aedes aegypti fourth instar larvae were treated with 2.5 μM chlorpromazine for two hours to inhibit clathrin, then rinsed in water for 15 minutes, and then soaked in small groups of five in different dsRNA lengths and structures targeting the snf7 gene's transcripts for three hours. The larvae were then transferred to water containing food, and after 30 hours, larvae were sacrificed, RNA extracted, and qRT-PCR was used to measure snf7 transcript levels. Table 6 shows the results of RNAi-mediated knockdown of snf7 by alternative dsRNA molecules in A. aegypti larvae treated with chlorpromazine. Value represent the mean and standard deviations of 5 replicate experiments of pools of 5 larvae. Treatments with significant knockdown, even in the presence of chlorpromazine, are highlighted with an asterisk.

As can be seen in Table 6, chlorpromazine treatment completely abolished transcript knockdown mediated by long dsRNA, siRNA, long and short and hpRNAs. Larvae treated with pcRNA demonstrated strong snf7 transcript knockdown, even when treated with chlorpromazine. These findings indicate that CME is the primary means of dsRNA entry into mosquito gut cells, but that pcRNA is able to enter the gut cells independently of CME mechanisms.

### Example 5—Treatment of Spodoptera Larvae with pcRNA Induces RNAi, Whereas Other dsRNA Structures Fail

First instar Spodoptera frugiperda larvae were starved for 12 hours and then droplet fed different dsRNAs targeting the actin gene. The insects were transferred to an artificial diet for 24 hours, and were then sacrificed, RNA extracted, and qRT-PCR was used to assess actin transcript levels. Table 7 shows the results of the RNAi-mediated knockdown of actin transcripts by alternative dsRNA molecules in first instar S. frugiperda larvae. The values represent the means and standard errors for 15 individual insects.

As can be seen in Table 7, linear dsRNAs (long or short) and hairpin RNAs (long and short) failed to induce knockdown, but larvae that fed short pcRNAs showed significant 52% knockdown of actin transcripts. These findings indicate the pcRNAs can mediate RNAi in an dsRNA insensitive insect.

While the preferred embodiments of the invention have been described above, it will be recognized and understood that various modifications may be made therein, and the appended claims are intended to cover all such modifications which may fall within the spirit and scope of the invention.

### REFERENCES

1. Gu L, Knipple DC (2013) Recent advances in RNA interference research in insects: Implications for future insect pest management strategies. Crop Prot. 45, 36-40.
2. Baum JA et al. (2007) Control of coleopteran insect pests through RNA interference. Nat. Biotechnol. 25, 1322-1326.
3. Whyard S, Singh AD, Wong S. (2009) Ingested double-stranded RNAs can act as species-specific insecticides. Insect Biochem. Mol. Biol. 39, 824-832.
4. Baum JA, Roberts JK. (2014) Progress towards RNAimediated insect pest management. Adv. Insect Physiol. 47, 249-295.
5. Shukla JN, Kalsi M, Sethi A, Narva KE, Fishilevich E, Singh S, Mogilicherla K, Palli SR. (2016) Reduced stability and intracellular transport of dsRNA contribute to poor RNAi response in lepidopteran insects. RNA Biol. 13, 656-669.
6. Khajuria C, et al. (2018) Development and characterization of the first dsRNA-resistant insect population from western corn rootworm, Diabrotica virgifera virgifera LeConte. PLoS One 13, 1-19.
7. Velez AM, Fishilevich E (2018) The mysteries of insect RNAi: a focus on dsRNA uptake and transport. Pestic Biochem Physiol. 151, 25-31.
8. Zhuang JJ, Hunter CP (2012) RNA interference in Caenorhabditis elegans: Uptake, mechanism, and regulation. Parasitology 139, 560-573.
9. Pinheiro DH, et al. (2018) Clathrin-dependent endocytosis is associated with RNAi response in the western corn rootworm, Diabrotica virgifera virgifera LeConte. PLoS One 13, 1-19.
10. Tomoyasu Y, et al. (2008) Exploring systemic RNA interference in insects: A genome-wide survey for RNAi genes in Tribolium. Genome Biol. 9, 1-22.
11. Saleh MC, et al. (2006) The endocytic pathway mediates cell entry of dsRNA to induce RNAi silencing. Nat. Cell Biol. 8, 793-802.
12. Ivashuta S, et al. (2015) Environmental RNAi in herbivorous insects. RNA 21, 840-850.
13. Mysore K, et al. (2019) Characterization of a broad-based mosquito yeast interfering RNA larvicide with a conserved target site in mosquito semaphorin-1a genes. Parasites and Vectors 12, 1-15.
14. Bartoletti R, et al. (2017) Short hairpin RNA is more effective than long hairpin RNA in eliciting pointed loss-offunction phenotypes in Drosophila. genesis.

2017;55:e23036
15. MacRae IJ, Zhou K, Doudna JA, Structural determinants of RNA recognition and cleavage by Dicer. Nat. Struct. Mol. Biol. 14, 934-940 (2007).

TABLE 1
List of dsRNAs used in this study.

| | | Double- |
|---|---|---|
| | | stranded |
| Gene | DsRNA sequences | length |
| Aedes Snf7 | Derived from Vectorbase Accession number: AAEL001698 | |
| Long-dsRNA | AGCTGAGGGACATCGAGAATATGCTGACCAAAAAGCAGGAAT | 204 bp |
| SEQ ID No. 1 | TCCTGGAAAAAAAGATCGAAGTTGAATTGGACACTGCGCGAAA | |
| | GAACGGAACCAAAAACAAGAGAGCTGCACTTCAGGCACTCAA | |
| | ACGAAAGAAGCGATATGAGAAACAACTGACTCAGATCGATGG | |
| | CACTTTGTCCACGATTGAAATGCAACGGGAAGCCT | |
| SIRNA | AGATTCAAATTGAGATTGAAGAC | 23 bp |
| SEQ ID No. 2 | | |
| Long hpRNA | AGCTGAGGGACATCGAGAATATGCTGACCAAAAAGCAGGAAT | 106 bp |
| SEQ ID No. 3 | TCCTGGAAAAAAAGATCGAAGTTGAATTGGACACTGCGCGAAA | |
| | GAACGGAACCAAAAACAAGAGAAAAAAAAACTCTTGTTTTTGG | |
| | TTCCGTTCTTTCGCGCAGTGTCCAATTCAACTTCGATCTTTTTT | |
| | TCCAGGAATTCCTGCTTTTTGGTCAGCATATTCTCGATGTCCCT | |
| | CAGCT | |
| shRNA | AGATTCAAATTGAGATTGAAGACAAAAAAAAAGTCTTCAATCTC | 23 bp |
| SEQ ID No. 4 | AATTTGAATCT | |
| Long pcRNA | AGCTGAGGGACATCGAGAATATGCTGACCAAAAAGCAGGAAT | 106 bp |
| SEQ ID No. 5 | TCCTGGAAAAAAAGATCGAAGTTGAATTGGACACTGCGCGAAA | |
| | GAACGGAACCAAAAACAAGAGAAAAAAAAACTCTTGTTTTTGG | |
| | TTCCGTTCTTTCGCGCAGTGTCCAATTCAACTTCGATCTTTTTT | |
| | TCCAGGAATTCCTGCTTTTTGGTCAGCATATTCTCGATGTCCCT | |
| | CAGCTTCAGAAAAAAAAACAGA | |
| Short pcRNA | AGATTCAAATTGAGATTGAAGACAAAAAAAAAGTCTTCAATCTC | 28 bp |
| SEQ ID No. 6 | AATTTGAAT CTTATCTAAAAAAAAAAGATA | |
| Aedes kermit | Derived from Vectorbase Accession number: AAEL012658 | |
| Long-dsRNA | CTACGAGAATAACAACGGCTTTAACGGCAAGAATCTGGCAGG | 213 bp |
| SEQ ID No. 7 | GAGCACAATGCCCACGGCACCGCACATCGATGACCTTAACGG | |
| | GAGCCACGAGGCCAACGTGACCAAGCCACAGCTCGTGTTCAA | |
| | CTGCCAGCTGGCCCACGGCAGTCCAACGGGCTTCATCACCGG | |
| | GTTTGCCAGCGTCAAGGAGCTGTACCAGAAGATCGCCGAATG | |
| | CTA | |
| SIRNA | GATACGACACTTCATGGAATGTT | 23 bp |
| SEQ ID No. 8 | | |
| Long hairpin | CTACGAGAATAACAACGGCTTTAACGGCAAGAATCTGGCAGG | 106 bp |
| SEQ ID No. 9 | GAGCACAATGCCCACGGCACCGCACATCGATGACCTTAACGG | |
| | GAGCCACGAGGCCAACGTGACCAAAAAAAAAAATTGGTCACG | |
| | TTGGCCTCGTGGCTCCCGTTAAGGTCATCGATGTGCGGTGCC | |
| | GTGGGCATTGTGCTCCCTGCCAGATTCTTGCCGTTAAAGCCGT | |
| | TGTTATTCTCGTAG | |
| shRNA | GATACGACACTTCATGGAATGTTAAAAAAAAAAACATTCCATGA | 23 bp |
| SEQ ID No. 10 | AGTGTCGT ATC | |
| Long pcRNA | CTACGAGAATAACAACGGCTTTAACGGCAAGAATCTGGCAGG | |
| SEQ ID No. 11 | GAGCACAATGCCCACGGCACCGCACATCGATGACCTTAACGG | |
| | GAGCCACGAGGCCAACGTGACCAAAAAAAAAAATTGGTCACG | |
| | TTGGCCTCGTGGCTCCCGTTAAGGTCATCGATGTGCGGTGCC | |
| | GTGGGCATTGTGCTCCCTGCCAGATTCTTGCCGTTAAAGCCGT | |
| | TGTTATTCTCGTAGAAAAGAAAAAAAAACTTTT | |
| pcRNA | GATACGACACTTCATGGAATGTTAAAAAAAAAAACATTCCATGA | 28 bp |
| SEQ ID No. 12 | AGTGTCGTA TCAGAATAAAAAAAAAATTCT | |
| Spodoptera | | |
| actin | Derived from NCBI accession: KY231202.1 | |
| Long dsRNA | CGTTCGTGACATCAAGGAGAAGCTGTGCTATGTCGCCCTCGA | 200 bp |
| SEQ ID No. 13 | CTTCGAGCAGGAGATGGCCACCGCTGCCGCCTCCACCTCCCT | |
| | CGAGAAGTCCTACGAACTTCCCGACGGTCAGGTCATCACCAT | |
| | CGGTAACGAGAGGTTCCGTTGCCCTGAAGCCCTCTTCCAGCC | |
| | TTCCTTCTTGGGTATGGAATCTTGCGGTATCC | |
| SIRNA | CGTTCGTGACATCAAGGAGAA | 23 bp |
| SEQ ID No. 14 | | |
| Long hairpin | CGTTCGTGACATCAAGGAGAAGCTGTGCTATGTCGCCCTCGA | 108 bp |
| SEQ ID No. 15 | CTTCGAGCAGGAGATGGCCACCGCTGCCGCCTCCACCTCCCT | |
| | CGAGAAGTCCTACGAACTTCCCGACAAAAAAAAAGTCGGGAA | |
| | GTTCGTAGGACTTCTCGAGGGAGGTGGAGGCGGCAGCGGTG | |
| | GCCATCTCCTGCTCGAAGTCGAGGGCGACATAGCACAGCTTC | |
| | TCCTTGATGTCACGAACG | |
| Short hpRNA | CGTTCGTGACATCAAGGAGAAAAAAAAAAATTCTCCTTGATGT | 23 bp |
| SEQ ID No. 16 | CACGAACG | |
| Long pcRNA | CGTTCGTGACATCAAGGAGAAGCTGTGCTATGTCGCCCTCGA | 108 bp |
| SEQ ID No. 17 | CTTCGAGCAGGAGATGGCCACCGCTGCCGCCTCCACCTCCCT | |
| | CGAGAAGTCCTACGAACTTCCCGACAAAAAAAAAGTCGGGAA | |
| | GTTCGTAGGACTTCTCGAGGGAGGTGGAGGCGGCAGCGGTG | |
| | GCCATCTCCTGCTCGAAGTCGAGGGCGACATAGCACAGCTTC | |
| | TCCTTGATGTCACGAACGATTTCAAAAAAAAAGAAAT | |
| Short pcRNA | CGTTCGTGACATCAAGGAGAAAAAAAAAAATTCTCCTTGATGT | 23 bp |
| SEQ ID No. 18 | CACGAACGATTTCAAAAAAAAAGAAAT | |
| | Derived from: | |
| Spodoptera | genome_corn_v3.0_spodoptera_frugiperda_v3.0: scaffold | |
| kermit | 3463 | |
| Long dsRNA | AATCAACAGTTTGCTGGAATCGTTCATGGGCATCAACGACTCC | 205 bp |
| SEQ ID No. 19 | GAGTTAGCATCTCAGATGTGGGATCTTGCCGAAGGAAAAGCA | |
| SIRNA | AATTCTATGCAACTAGCTGAAGCCATCGATAATAGTGACTTACA | |
| SEQ ID No. 20 | AGAATTCGGATTCACTGACGAGTTTATAATTGAATTATGGGGT | |
| | GTTATTACTGACGCTAGATCTGGTAGACTCAGT | |
| | ACGCTAGATCTGGTAGACTCAGT | |
| Long hairpin | AATCAACAGTTTGCTGGAATCGTTCATGGGCATCAACGACTCC | 23 bp |
| SEQ ID No. 21 | GAGTTAGCATCTCAGATGTGGGATCTTGCCGAAGGAAAAGCA | |
| | AATTCTATGCAACTAGCTGAAGCCAAAAAAAAAGGCTTCAGCT | 205 bp |
| | AGTTGCATAGAATTTGCTTTTCCTTCGGCAAGATCCCACATCT | |
| | GAGATGCTAACTCGGAGTCGTTGATGCCCATGAACGATTCCA | |
| | GCAAACTGTTGATT | |
| short hp | ACGCTAGATCTGGTAGACTCAGTAAAAAACTGAGTCTACCAGA | 23 bp |
| SEQ ID No. 22 | TCTAGCGGT | |
| Long pcRNA | AATCAACAGTTTGCTGGAATCGTTCATGGGCATCAACGACTCC | 205 bp |
| SEQ ID No. 23 | GAGTTAGCATCTCAGATGTGGGATCTTGCCGAAGGAAAAGCA | |
| | AATTCTATGCAACTAGCTGAAGCCAAAAAAAAAGGCTTCAGCT | |
| | AGTTGCATAGAATTTGCTTTTCCTTCGGCAAGATCCCACATCT | |
| | GAGATGCTAACTCGGAGTCGTTGATGCCCATGAACGATTCCA | |
| | GCAAACTGTTGATTTTCTAAAAAAAAAAGAA | |
| Short pcRNA | ACGCTAGATCTGGTAGACTCAGTAAAAAACTGAGTCTACCAGA | 23 bp |
| SEQ ID No. 24 | TCTAGCGTTTCTAAAAAAAAAAGAA | |

TABLE 2
Knockdown of two genes using different lengths and structured
dsRNAs in Ae. aegypti CCL125 cells using Lipofectamine ™(3000)
transfection reagent to deliver the dsRNAs to the cells.
The long dsRNAs targeting Snf7 and kermit were 204 and 213
bp, respectively, whereas the siRNA, short hpRNA, and short
pcRNA each contained a contiguous 23 bp long dsRNA section.
Hence, the cells were treated with 10x higher doses of the
short dsRNAs to provide the cells with equivalent numbers
of Dicer-generated (in vivo) siRNAs. The values represent
the means and standard errors of 10 replicate well treatments.
No significant difference in % knockdown was observed for
any of the different dsRNA treatments (ANOVA, p > .7
for all comparisons).

| Target Gene | dsRNA (dose/well) | % Knockdown |
|---|---|---|
| Snf7 | Long (20 pmol) | 91 ± 3 |
| | siRNA (200 pmol) | 88 ± 4 |
| | Long hpRNA (20 pmol) | 92 ± 5 |
| | shRNA (200 pmol) | 93 ± 2 |
| | Long pcRNA | 93 ± 6 |
| | pcRNA (200 pmol) | 94 ± 3 |
| Kermit | Long (20 pmol) | 88 ± 5 |
| | siRNA (200 pmol) | 86 ± 4 |
| | Long hpRNA | 84 ± 5 |
| | shRNA (200 pmol) | 89 ± 3 |
| | Long pcRNA | 94 ± 4 |
| | pcRNA (200 pmol) | 91 ± 4 |

TABLE 3
Knockdown of two genes in Spodoptera Sf9 cells when different
dsRNAs are delivered by transfection reagents. All dsRNA molecules
were equally effective (when doses were adjusted to accommodate
different numbers of siRNA generated by the long dsRNAs).
All values represent the means and standard errors from 10
replicate wells of cells. The extent of knockdown was similar
for all treatments (ANOVA, p > 0.5)

| Target Gene | dsRNA (dose/well) | % Knockdown |
|---|---|---|
| actin | Long (20 pmol) | 83 ± 5 |
| | siRNA (200 pmol) | 80 ± 6 |
| | Long hpRNA (20 pmol) | 86 ± 7 |
| | shRNA (200 pmol) | 79 ± 5 |
| | Long pcRNA | 83 ± 5 |
| | pcRNA (200 pmol) | 78 ± 5 |
| kermit | Long (20 pmol) | 78 ± 6 |
| | siRNA (200 pmol) | 73 ± 5 |
| | Long hpRNA | 74 ± 6 |
| | shRNA (200 pmol) | 72 ± 6 |
| | Long pcRNA | 74 ± 6 |
| | pcRNA (200 pmol) | 77 ± 5 |

TABLE 4
Percent transcript knockdown of two target genes (snf7 or kermit) following exposure
of A. aegypti CCL-125 cells to different structured dsRNAs in the presence
or absence of chlorpromazine, an inhibitor of clathrin-mediated endocytosis.
Values represent the means and standard errors of 5 replicate experiments consisting
of 6 well each. Treatments demonstrating significantly impaired RNAi, relative
to the “no inhibit control” are highlighted with an asterisk.

| DSRNA | | | | |
|---|---|---|---|---|
| TREATMENT | NO INHIBITOR | +CPZ | +CYTO-D | +MBC |
| SNF7 | | | | |
| NO DSRNA | 0.00 ± 2.35 | — | — | — |
| LONG-DSRNA | 86.65 ± 1.25 | 8.22 ± 2.59* | 84.45 ± 2.31 | 83.89 ± 2.43 |
| SIRNA | 25.14 ± 2.17 | 11.56 ± 2.11* | 26.34 ± 2.42 | 24.98 ± 2.16 |
| LONG HPRNA | 88.37 ± 2.36 | 13.22 ± 3.24* | 84.76 ± 2.07 | 86.94 ± 2.16 |
| SHRNA | 32.40 ± 2.23 | 12.98 ± 2.99* | 33.03 ± 2.47 | 31.86 ± 2.78 |
| LONG PCRNA | 89.56 ± 2.09 | 85.43 ± 2.46 | 85.89 ± 2.19 | 87.56 ± 2.44 |
| PCRNA | 82.64 ± 2.34 | 74.58 ± 2.83 | 82.49 ± 2.66 | 81.77 ± 3.15 |
| KERMIT | | | | |
| NO DSRNA | 0.00 ± 2.10 | — | — | — |
| LONG DSRNA | 76.73 ± 1.68 | 9.27 ± 2.63* | 75.53 ± 2.06 | 77.83 ± 2.23 |
| SIRNA | 38.64 ± 2.47 | 4.86 ± 1.54* | 37.09 ± 3.28 | 39.53 ± 2.97 |
| LONG HPRNA | 79.44 ± 2.82 | 6.41 ± 2.03* | 78.51 ± 1.98 | 80.46 ± 3.12 |
| SHRNA | 65.02 ± 1.52 | 9.96 ± 1.43* | 67.22 ± 3.11 | 64.91 ± 2.47 |
| LONG PCRNA | 80.23 ± 3.38 | 74.21 ± 4.51 | 80.01 ± 3.26 | 79.88 ± 2.93 |
| PCRNA | 75.42 ± 1.85 | 66.90 ± 1.90 | 76.37 ± 2.44 | 74.28 ± 3.17 |

TABLE 5
Percent knockdown of actin transcripts in Sf9 cells treated
with different dsRNAs. The values represent the means
and averages of 5 replicates of 4 wells of cells.

| | ACTIN | SIGNIFICANT FROM |
|---|---|---|
| | KNOCKDOWN | NEGATIVE CONTROL? |
| DSRNA | (%) | (Y/N) |
| NONE (NEG. | 0.00 ± 4.22 | — |
| CONTROL) | | |
| LONG DSRNA | 4.25 ± 3.07 | N |
| SIRNA | 0.12 ± 3.36 | N |
| LONG HPRNA | 4.55 ± 3.72 | N |
| SHORT HPRNA | 4.86 ± 3.60 | N |
| LONG PCRNA | 17.36 ± 3.81 | Y |
| SHORT PCRNA | 51.21 ± 4.63 | Y |

TABLE 6
RNAi-mediated knockdown of snf7 by alternative dsRNA molecules in
A. aegypti larvae treated with chlorpromazine. Value represent
the mean and standard deviations of 5 replicate experiments of pools
of 5 larvae. Treatments with significant knockdown, even in the
presence of chlorpromazine are highlighted with an asterisk.

| DSRNA TREATMENT | −CPZ | +CPZ |
|---|---|---|
| NO DSRNA | 0.00 ± 3.97 | 2.66 ± 3.01 |
| LONG-DSRNA | 59.42 ± 3.09 | 4.98 ± 4.10 |
| SIRNA | 17.12 ± 4.83 | 1.30 ± 3.02 |
| LONG HPRNA | 64.27 ± 4.12 | 3.44 ± 2.32 |
| SHORT HPRNA | 41.40 ± 5.24 | 5.00 ± 3.94 |
| LONG PCRNA | 64.94 ± 4.21 | 41.38 ± 5.46* |
| PCRNA | 45.82 ± 5.30 | 33.04 ± 4.66* |

TABLE 7
RNAi-mediated knockdown of actin transcripts by alternative dsRNA
molecules in first instar S. frugiperda larvae. The values represent
the means and standard errors for 15 individual insects.

| | | SIGNIFICANTLY |
|---|---|---|
| | PERCENT | DIFFERENT FROM |
| | ACTIN | NEGATIVE CONTROL |
| DSRNA TREATMENT | KNOCKDOWN | (YES/NO) |
| NO DSRNA (NEG. | 0.00 ± 5.62 | — |
| CONT.) | | |
| LONG-DSRNA | 4.01 ± 4.33 | N |
| SIRNA | 2.34 ± 2.01 | N |
| LONG HPRNA | 6.55 ± 4.83 | N |
| SHORT HPRNA | 3.79 ± 4.04 | N |
| LONG PCRNA | 24.36 ± 7.44 | Y |
| SHORT PCRNA | 52.2. ± 8.23 | Y |

1. (canceled)

2. (canceled)

3. (canceled)

4. (canceled)

5. (canceled)

6. (canceled)

7. (canceled)

8. (canceled)

9. (canceled)

10. A method of reducing feeding damage to a plant from a biological pest of interest comprising:
applying to the plant to be protected from the biological pest of interest an effective amount of a synthetic RNA molecule to at least a portion of the plant to be protected from the biological pest of interest, the synthetic RNA molecule folding to form a secondary structure following synthesis that comprises:
a) a double stranded region comprising an RNAi sequence specific for the biological pest of interest, said RNAi sequence being at least 21 nucleotides;
b) two single-stranded hairpin loops flanking either end of the double-stranded region; and
c) a 5′ end and a 3′ end within the double stranded region that are closed but not covalently sealed,
said synthetic RNA molecule reducing feeding damage to the plant to be protected by reducing feeding activity of the biological pest of interest following ingestion of said synthetic RNA molecule by the insect of interest.

11. The method according to claim 10 wherein the synthetic RNA molecule folds spontaneously.

12. The method according to claim 10 wherein in the folded RNA molecule, the 5′ end and the 3′ end are separated by a gap corresponding to the length of at least one phosphodiester bond.

13. The method according to claim 12 wherein the length of the double stranded RNA region is at least 23 nucleotides on a 5′ side of the gap.

14. The method according to claim 13 wherein the length of the double stranded region is at least 3 nucleotides on a 3′ side of the gap.

15. The method according to claim 10 wherein each hairpin loop independently comprises 3-15 nucleotides.

16. (canceled)

17. (canceled)

18. (canceled)

19. The method according to claim 10 wherein the synthetic RNA molecule is co-administered with a nuclease inhibitor.

20. A method of protecting a plant from feeding damage from a biological pest of interest comprising:
applying to the plant to be protected from the biological pest of interest an effective amount of a synthetic RNA molecule to at least a portion of the plant to be protected from the biological pest of interest, the synthetic RNA molecule folding to form a secondary structure following synthesis that comprises:
a) a double stranded region comprising an RNAi sequence specific for the biological pest of interest, said RNAi sequence being at least 21 nucleotides;
b) two single-stranded hairpin loops flanking either end of the double-stranded region; and
c) a 5′ end and a 3′ end within the double stranded region that are closed but not covalently sealed,
said synthetic RNA molecule reducing feeding damage to the plant to be protected by reducing feeding activity of the biological pest of interest following ingestion of said synthetic RNA molecule by the insect of interest.

21. The method according to claim 20 wherein the synthetic RNA molecule folds spontaneously.

22. The method according to claim 20 wherein in the folded RNA molecule, the 5′ end and the 3′ end are separated by a gap corresponding to the length of at least one phosphodiester bond.

23. The method according to claim 22 wherein the length of the double stranded RNA region is at least 23 nucleotides on a 5′ side of the gap.

24. The method according to claim 23 wherein the length of the double stranded region is at least 3 nucleotides on a 3′ side of the gap.

25. The method according to claim 20 wherein each hairpin loop independently comprises 3-15 nucleotides.

26. (canceled)

27. (canceled)

28. (canceled)

29. The method according to claim 20 wherein the synthetic RNA molecule is co-administered with a nuclease inhibitor.

30. A method of killing a biological pest of interest comprising:
applying to at least a portion of a food source of the biological pest of interest an effective amount of a synthetic RNA molecule, the synthetic RNA molecule folding to form a secondary structure following synthesis that comprises:
a) a double stranded region comprising an RNAi sequence specific for the biological pest of interest, said RNAi sequence being at least 21 nucleotides;
b) two single-stranded hairpin loops flanking either end of the double-stranded region; and
c) a 5′ end and a 3′ end within the double stranded region that are closed but not covalently sealed,
said synthetic RNA molecule killing the biological pest of interest following ingestion of said synthetic RNA molecule by the biological pest of interest.

31. The method according to claim 30 wherein the synthetic RNA molecule folds spontaneously.

32. The method according to claim 30 wherein in the folded RNA molecule, the 5′ end and the 3′ end are separated by a gap corresponding to the length of at least one phosphodiester bond.

33. The method according to claim 32 wherein the length of the double stranded RNA region is at least 23 nucleotides on a 5′ side of the gap.

34. The method according to claim 33 wherein the length of the double stranded region is at least 3 nucleotides on a 3′ side of the gap.

35. The method according to claim 30 wherein each hairpin loop independently comprises 3-15 nucleotides.

36. (canceled)

37. (canceled)

38. (canceled)

39. The method according to claim 30 wherein the synthetic RNA molecule is co-administered with a nuclease inhibitor.


SEQUENCE LISTING (24 sequences)

SEQ ID NO: 1 - DNA, 204 bp, Artificial
  Double stranded RNA
agctgagggacatcgagaatatgctgaccaaaaagcaggaattcctggaaaaaaagatcgaagttgaattggacactgcgcgaaagaacggaaccaaaaacaagagagctgcacttcaggcactcaaacgaaagaagcgatatgagaaacaactgactcagatcgatggcactttgtccacgattgaaatgcaacgggaagcct

SEQ ID NO: 2 - DNA, 23 bp, Artificial
  short interfering RNA
agattcaaattgagattgaagac

SEQ ID NO: 3 - DNA, 221 bp, Artificial
  hairpin RNA sequence
agctgagggacatcgagaatatgctgaccaaaaagcaggaattcctggaaaaaaagatcgaagttgaattggacactgcgcgaaagaacggaaccaaaaacaagagaaaaaaaaactcttgtttttggttccgttctttcgcgcagtgtccaattcaacttcgatctttttttccaggaattcctgctttttggtcagcatattctcgatgtccctcagct

SEQ ID NO: 4 - DNA, 55 bp, artificial
  short hairpin RNA sequence
agattcaaattgagattgaagacaaaaaaaaagtcttcaatctcaatttgaatct

SEQ ID NO: 5 - DNA, 238 bp, Artificial
  Long paperclip RNA sequence
agctgagggacatcgagaatatgctgaccaaaaagcaggaattcctggaaaaaaagatcgaagttgaattggacactgcgcgaaagaacggaaccaaaaacaagagaaaaaaaaactcttgtttttggttccgttctttcgcgcagtgtccaattcaacttcgatctttttttccaggaattcctgctttttggtcagcatattctcgatgtccctcagcttcagaaaaaaaaacaga

SEQ ID NO: 6 - DNA, 74 bp, Artificial
  Short paperclip RNA sequence
agattcaaattgagattgaagacaaaaaaaaagtcttcaatctcaatttgaatcttatctaaaaaaaaaagata

SEQ ID NO: 7 - DNA, 213 bp, Artificial
  Long double stranded RNA sequence
ctacgagaataacaacggctttaacggcaagaatctggcagggagcacaatgcccacggcaccgcacatcgatgaccttaacgggagccacgaggccaacgtgaccaagccacagctcgtgttcaactgccagctggcccacggcagtccaacgggcttcatcaccgggtttgccagcgtcaaggagctgtaccagaagatcgccgaatgcta

SEQ ID NO: 8 - DNA, 23 bp, Artificial
  short interfering RNA structure
gatacgacacttcatggaatgtt

SEQ ID NO: 9 - DNA, 225 bp, Artificial
  Long Hairpin structure
ctacgagaataacaacggctttaacggcaagaatctggcagggagcacaatgcccacggcaccgcacatcgatgaccttaacgggagccacgaggccaacgtgaccaaaaaaaaaaattggtcacgttggcctcgtggctcccgttaaggtcatcgatgtgcggtgccgtgggcattgtgctccctgccagattcttgccgttaaagccgttgttattctcgtag

SEQ ID NO: 10 - DNA, 55 bp, Artificial
  Short Hairpin RNA structure
gatacgacacttcatggaatgttaaaaaaaaaaacattccatgaagtgtcgtatc

SEQ ID NO: 11 - DNA, 244 bp, Artificial
  Long pcRNA structure
ctacgagaataacaacggctttaacggcaagaatctggcagggagcacaatgcccacggcaccgcacatcgatgaccttaacgggagccacgaggccaacgtgaccaaaaaaaaaaattggtcacgttggcctcgtggctcccgttaaggtcatcgatgtgcggtgccgtgggcattgtgctccctgccagattcttgccgttaaagccgttgttattctcgtagaaaagaaaaaaaaactttt

SEQ ID NO: 12 - DNA, 244 bp, Artificial; identical to SEQ ID NO: 11
  Paperclip RNA structure

SEQ ID NO: 13 - DNA, 200 bp, Artificial
  Long double stranded RNA structure
cgttcgtgacatcaaggagaagctgtgctatgtcgccctcgacttcgagcaggagatggccaccgctgccgcctccacctccctcgagaagtcctacgaacttcccgacggtcaggtcatcaccatcggtaacgagaggttccgttgccctgaagccctcttccagccttccttcttgggtatggaatcttgcggtatcc

SEQ ID NO: 14 - DNA, 21 bp, Artificial
  short interfering RNA structure
cgttcgtgacatcaaggagaa

SEQ ID NO: 15 - DNA, 227 bp, Artificial
  Long RNA Hairpin forming structure
cgttcgtgacatcaaggagaagctgtgctatgtcgccctcgacttcgagcaggagatggccaccgctgccgcctccacctccctcgagaagtcctacgaacttcccgacaaaaaaaaagtcgggaagttcgtaggacttctcgagggaggtggaggcggcagcggtggccatctcctgctcgaagtcgagggcgacatagcacagcttctccttgatgtcacgaacg

SEQ ID NO: 16 - DNA, 51 bp, Artificial
  Short hairpin forming RNA structure
cgttcgtgacatcaaggagaaaaaaaaaaattctccttgatgtcacgaacg

SEQ ID NO: 17 - DNA, 246 bp, Artificial
  Long paperclip forming RNA structrure
cgttcgtgacatcaaggagaagctgtgctatgtcgccctcgacttcgagcaggagatggccaccgctgccgcctccacctccctcgagaagtcctacgaacttcccgacaaaaaaaaagtcgggaagttcgtaggacttctcgagggaggtggaggcggcagcggtggccatctcctgctcgaagtcgagggcgacatagcacagcttctccttgatgtcacgaacgatttcaaaaaaaaagaaat

SEQ ID NO: 18 - DNA, 70 bp, Artificial
  Short paperclip forming RNA
cgttcgtgacatcaaggagaaaaaaaaaaattctccttgatgtcacgaacgatttcaaaaaaaaagaaat

SEQ ID NO: 19 - DNA, 205 bp, Artificial
  Long double stranded RNA forming structure
aatcaacagtttgctggaatcgttcatgggcatcaacgactccgagttagcatctcagatgtgggatcttgccgaaggaaaagcaaattctatgcaactagctgaagccatcgataatagtgacttacaagaattcggattcactgacgagtttataattgaattatggggtgttattactgacgctagatctggtagactcagt

SEQ ID NO: 20 - DNA, 23 bp, Artificial
  short interfering RNA
acgctagatctggtagactcagt

SEQ ID NO: 21 - DNA, 227 bp, Artificial
  Long hairpin forming structure
aatcaacagtttgctggaatcgttcatgggcatcaacgactccgagttagcatctcagatgtgggatcttgccgaaggaaaagcaaattctatgcaactagctgaagccaaaaaaaaaggcttcagctagttgcatagaatttgcttttccttcggcaagatcccacatctgagatgctaactcggagtcgttgatgcccatgaacgattccagcaaactgttgatt

SEQ ID NO: 22 - DNA, 52 bp, Artificial
  Short hairpin RNA structure
acgctagatctggtagactcagtaaaaaactgagtctaccagatctagcggt

SEQ ID NO: 23 - DNA, 244 bp, Artificial
  Long paperclip forming RNA structure
aatcaacagtttgctggaatcgttcatgggcatcaacgactccgagttagcatctcagatgtgggatcttgccgaaggaaaagcaaattctatgcaactagctgaagccaaaaaaaaaggcttcagctagttgcatagaatttgcttttccttcggcaagatcccacatctgagatgctaactcggagtcgttgatgcccatgaacgattccagcaaactgttgattttctaaaaaaaaaagaa

SEQ ID NO: 24 - DNA, 68 bp, Artificial
  Short paperclip forming RNA structure
acgctagatctggtagactcagtaaaaaactgagtctaccagatctagcgtttctaaaaaaaaaagaa